(19) United States
(12) Patent Application Publication
(10) Pub. No.: US 2010/0249050 A1
(43) Pub. Date: Sep. 30, 2010

(54) EPOTHILONE GLYCOSIDE AND USE THEREOF

(75) Inventors: Yuezhong LI, Jinan (CN); Yuemao SHEN, Jinan (CN); Lin ZHAO, Jinan (CN); Chunhua LU, Jinan (CN)

Correspondence Address: MATTHIAS SCHOLL, 14781 MEMORIAL DRIVE, SUITE 1319, HOUSTON, TX, 77079, US

(73) Assignee: SHANDONG UNIVERSITY, Jinan (CN)

(21) Appl. No.: 12/796,700

(22) Filed: Jun. 9, 2010

Related U.S. Application Data
(63) Continuation of application No. PCT/CN2008/001946, filed on Nov. 28, 2008.

(30) Foreign Application Priority Data
Oct. 6, 2008 (CN) 200810157514.2

(51) Int. Cl. A61K 31/7048; C07H 17/08; A61P 35/00

(52) U.S. Cl. 514/30; 536/7.1

(57) ABSTRACT

An epothilone glycoside having a formula of

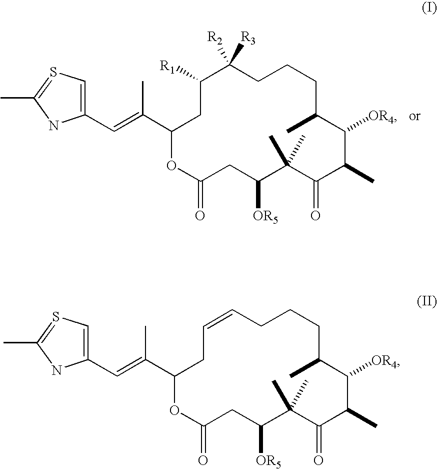

and a pharmaceutical composition having the epothilone glycoside and a pharmaceutically acceptable excipient. The epothilone glycoside or the pharmaceutical composition having the epothilone glycoside can prevent or treat cancers such as liver cancer, lung cancer, and breast cancer.

### CROSS-REFERENCE TO RELATED APPLICATIONS

This application is a continuation of International Patent Application No. PCT/CN2008/001946 with an international filing date of Nov. 28, 2008, designating the United States, now pending, and further claims priority benefits to Chinese Patent Application No. 200810157514.2 filed Oct. 6, 2008. The contents of all of the aforementioned applications, including any intervening amendments thereto, are incorporated herein by reference

### BACKGROUND OF THE INVENTION

1. Field of the Invention

The invention relates to a compound, a method of preparing the same, as well as a use thereof, and more particularly to a compound of epothilone glycoside, and a use thereof as active ingredient for the treatment of cancers.

2. Description of the Related Art

Sorangium cellulosum (myxobacteriales) can produce a large variety of secondary metabolites including epothilone. Epothilone is a macrolide compound with antitumor activity, among which epothilone A and epothilone B are common, having a formula of

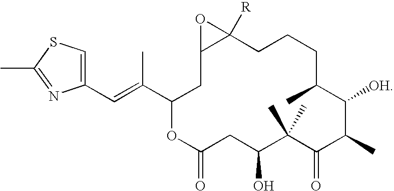

(R═H, epothilone A; R═CH₃, epothilone B)

In 1993, Natural Epothilone Exhibiting Cytotoxicity was Isolated by Rechenbach and Hale. In 1995, Merch Research Laboratories verified epothilone had the similar mechanism to pacilitaxel (TAXOL®). In 1996, Hale et al. disclosed the three-dimensional chemical structure of epothilone B, in which epoxide/thiazole/ketone was involved, and thereby the compound was designated “epothilone”.

Similar to pacilitaxel (TAXOL®), epothilone can promote the polymerization and stability of microtubules, induce tubulin polymer to form a super-stable structure, inhibit mitosis, and thereby can prevent the proliferation of tumor cells. However, epothilone is much simpler than pacilitaxel in molecular structure, and has better water solubility and good potential for chemical modification. Additionally, epothilone exhibits high inhibitive activity on tumor cells that have strong resistance against pacilitaxel. Furthermore, due to isolation from Sorangium cellulosum, epothilone can be produced by large-scale fermentation.

Having the above-mentioned advantages, epothilone is viewed as a good substitute of pacilitaxel and has bright prospects for prevention and treatment of cancer.

Up to date, a variety of epothilone analogs have been in clinical evaluation and even on sale, including ixabepilone (azaepothilone B, codenamed BMS-247550, developed by Bristol-Myers Squibb), BMS-310705 (a water-soluble analog of epothilone B, developed by Bristol-Myers Squibb/GBF), patupilone (epothilone B, EP0906, developed by Novartis Pharma AG), epothilone R1645 (KOS-1584, developed by Roche and Kosan Biosciences Incorporated (Nasdaq: KOSN)), ZK-EPO, and C20-desmethyl-C20-methylsulfanyl-Epo B (ABJ879, Novartis Pharma AG). However there are no reports related to epothilone glycosides.

### SUMMARY OF THE INVENTION

In view of the above-described problems, it is one objective of the invention to provide an epothilone glycoside having anticancer activity.

It is another objective of the invention to provide a pharmaceutical composition comprising an epothilone glycoside having anticancer activity.

It is another objective of the invention to provide a method for the treatment and prevention of cancer.

To achieve the above objectives, in accordance with one embodiment of the invention, there is provided an epothilone glycoside having anticancer activity, the epothilone glycoside having a formula of

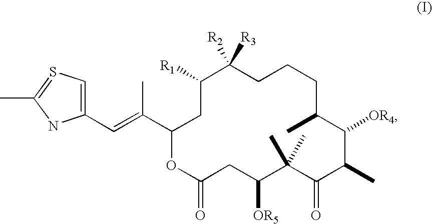

wherein

| | |
|---|---|
| R₁ + R₂ = O, R₃ = R₄ = H, R₅ = glycosyl | epothilone glycoside A-1 |
| R₁ + R₂ = O, R₃ = R₅ = H, R₄ = glycosyl | epothilone glycoside A-2 |
| R₁ + R₂ = O, R₃ = CH₃, R₄ = H, R₅ = glycosyl | epothilone glycoside B-1 |
| R₁ + R₂ = O, R₃ = CH₃, R₅ = H, R₄ = glycosyl | epothilone glycoside B-2, |

or

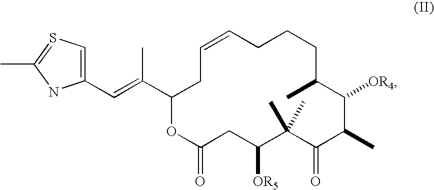

wherein

| | |
|---|---|
| R₄ = H, R₅ = glycosyl | epothilone glycoside C-1 |
| R₄ = glycosyl, R₅ = H | epothilone glycoside C-2. |

In a class of this embodiment, the epothilone glycoside is isolated from a solid or liquid fermentation product of Sorangium cellulosum So0157-2 CCTCC NO: M 208078, Sorangium cellulosum So0157-2 CCTCC NO: M 208078 being deposited in the China Center for Type Culture Collection (Wuhan University, Wuhan, China) on May 27, 2008.

In accordance with another embodiment of the invention, there is provided a pharmaceutical composition comprising a therapeutically effective amount of epothilone glycoside of formula (I) or (II) and a pharmaceutically acceptable excipient for the treatment and prevention of cancer.

In a class of this embodiment, the cancer is liver cancer.

In a class of this embodiment, the cancer is lung cancer.

In a class of this embodiment, the cancer is breast cancer.

In a class of this embodiment, the pharmaceutically acceptable excipient is a diluent such as water; a filler such as starch, sugar, and similar; an adhesive such as cellulose derivatives, alginate, gelatin, and polyvinylpyrrolidone; a wetting agent such as glycerol; a disintegrating agent such as agar, calcium carbonate, and sodium bicarbonate; an absorption enhancer such as quaternary ammonium compounds; a surface active agent such as hexadecanol; an adsorption carrier such as kaolin clay and bentonite; a lubricant such as talc, calcium stearate, magnesium, and polyethylene glycol. Other adjuvants such as flavor agents and sweeteners can also be added to the composition.

In another respect, the invention provides a method for the treatment and prevention of cancer comprising administering a patient in need thereof an epothilone glycoside of formula (I) or (II).

Studies have shown the epothilone glycoside of the invention can treat and prevent liver cancer, lung cancer, and breast cancer. At the concentration of 10⁻⁶ M, the compound has strong inhibition on human liver cancer cell HepG2, and exhibits a certain inhibition on human lung cancer cell A-549 and breast cancer cell MDA-MB-435, which shows the active site of the compound can function as a chemical inhibitor of liver cancer, lung cancer, and breast cancer. Thus, the compound or the pharmaceutical composition comprising, the compound can be used for the preparation of an anticancer medication.

In a class of this embodiment, an administration mode of the compound/pharmaceutical composition is oral administration, nasal inhalation, rectal administration, or parenteral administration.

In a class of this embodiment, for oral administration, the compound/pharmaceutical composition is a solid dosage form such as tablets, powders, granules, capsules, etc., or a liquid dosage form such as aqueous agents, oil-based suspension, syrup, and elixir agents.

In a class of this embodiment, for parenteral administration, the compound/pharmaceutical composition is a solution for injection, an aqueous agent, or an oil-based suspension.

Preferably, the dosage form is as tablets, coated tablets, capsules, suppositories, nasal sprays and injections, and more preferably, is a formulation released at a specific site of intestine.

In a class of this embodiment, the dosage form is produced by conventional methods of pharmaceutical field, for example, mixing the epothilone glycoside with one or more excipients, and then preparing as needed.

In a class of this embodiment, the epothilone glycoside accounts for between 0.1 and 99.5 wt. % of the pharmaceutical composition.

In a class of this embodiment, the epothilone glycoside accounts for between 0.5 and 95 wt. % of the pharmaceutical composition.

The effective amount of the compound is determined by administration mode, age and body weight of a patient, the type and severity of illness.

In a class of this embodiment, a daily dose of the epothilone glycoside is between 0.01 and 10 mg/Kg of body weight.

In a class of this embodiment, a daily dose of the epothilone glycoside is between 0.1 and 5 mg/Kg of body weight.

In a class of this embodiment, the epothilone glycoside can be administered once or several times.

### DETAILED DESCRIPTION OF THE EMBODIMENTS

For further illustrating the invention, experiments detailing an epothilone glycoside and a method of preparing the same are described below. It should be noted that the following examples are intended to describe and not to limit the invention.

### Example 1

### 1. Isolation of Strain, Deposition, Isolation and Purification of Epothilone Glycoside, and Antitumor Activity Test (Take Epothilone Glycoside A-1 as an Example)

The soil for screening bacteria was collected from the shore of Chenghai Lake, Yunan province, China. The screening method was as follows.

Sterile filter paper was placed on a CNST plate medium having 25 μg/mL sterilized cycloheximide. The medium pH 7.2 was cultured at 30° C. Growth of myxobacteria was observed daily under an anatomical lens and 2 days later, newly-produced myxobacteria were transferred to a CNST fresh medium pH 10.0 for culture and purification. The culture temperature was 30° C. By 5 days later, a fruiting body was observed and transferred to a sterilized E. coli trace in a WCX plate medium containing 250 μg/mL kanamycin sulfate to remove a large variety of bacteria. Finally, Sorangium cellulosum at edge of the colony was transferred to filter paper of another CNST plate (pH 10.0), and thereby the purification was achieved. The purified and mature fruiting bodies of myxobacteria on the plate were collected, transferred to sterile 1.5×3 cm filter paper, and preserved in a sterile tube in a dry state. Based on this method, Sorangium cellulosum So0157 which was alkali-resistant and could produce epothilone was obtained. The bacteria can be used as a starting strain for further selection.

As a starting strain, Sorangium cellulosum So0157 was further acclimation-induced by repeated solid-liquid continuous interval culture.

Specifically, Sorangium cellulosum So0157 was cultured on inverted plates in a CNST solid medium (pH 9.0) at 30° C. A fruiting body was observed 7 days later, and fresh cells at the edge of the colony were transferred to 100 mL of liquid medium of VY/2. The cells were cultured by shaking by rotation for 5 days at 30° C. and 200 rpm. 10 mL of the first round of fermentation broth was transferred to 90 mL of liquid medium of VY/2, and cultured under rotation for 5 days at 30° C. and 200 rpm. Subsequently, 10 mL of the second round of fermentation broth was transferred to 90 mL of liquid medium of VY/2, and cultured under rotation for 5 days at 30° C. and 200 rpm, ending the first process of acclimation-induction. 1 mL of the last round of fermentation broth was cultured on inverted plates in another CNST solid medium (pH 9.0) at 30° C. to initiate the next culture process. After several processes, the obtained strain was cultured under rotation, and by evaluating the growth state and determining the yield of epothilone, a high-yield strain of epothilone was obtained. The strain was identified as a myxobacteria, by the State Key Laboratory for Microbial Technology, Shandong University, and 16S rDNA sequence information thereof was published (DQ256394.1). The strain was deposited in the China Center for Type Culture Collection (Wuhan University, Wuhan, China) on May 27, 2008, under deposition information: Sorangium cellulosum So0157-2, CCTCC NO: M 208078.

A formula of the CNST medium was: KNO₃ 0.5 g/L, Na₂HPO₄ 0.25 g/L, MgSO₄.7H₂O 1 g/L, FeCl₃ 0.001%, trace element solution 1 ml/L, agar 1.5%, and the pH was adjusted as needed. The formula was sterilized, a plate medium was prepared, and sterile filter paper was placed on the plate after cooling solidification.

A formula of the WCX medium for purifying the strain was (by weight percent): CaCl₂.2H₂O 0.15%, agar 1.6%, the pH was adjusted with KOH to 7.0. After sterilization, 25 μg/mL sterilized cycloheximide was added. After forming a plate medium, living E. coli was inoculated on the surface by densely crossing so as to induce the formation of a fruiting body of myxobacteria. E. coli was cultured with conventional LB culture medium.

A formula of the VY/2 medium for acclimation-inducing the strain was (by weight percent): active yeast, 0.5%; CaCl₂ 0.08%; VB₁₂ 0.5 μg/mL, pH 9.0.

A formula of the trace element solution was MnCl₂.4H₂O 0.1 g/L, CoCl₂ 0.02 g/L, CuSO₄ 0.01 g/L, Na₂MoO₄.2H₂O 0.01 g/L, ZnCl₂ 0.02 g/L, LiCl 0.005 g/L, SnCl₂.2H₂O 0.005 g/L, H₃BO₃ 0.01 g/L, KBr 0.02 g/L, and KI 0.02 g/L.

Studies showed that Sorangium cellulosum So0157-2, CCTCC NO: M 208078 produced the epothilone compound. By methods of solid fermentation, LC-MS, and activity tracking, not only epothilone A/B/C was detected, but also epothilone glycoside A-1, A-2, B-1, B-2, C-1, and C-2 having antitumor activity were obtained.

The molecular ion peak of epothilone glycoside A-1, A-2, B-1, B-2, C-1, and C-2, which were glycosides of epothilone A/B/C was also detected with LC-MS.

1.1 The fermentation of Sorangium cellulosum So0157-2, CCTCC NO: M 208078 and the Extraction of a Compound

Sorangium cellulosum So0157-2, CCTCC NO: M 208078 was transferred from a solid medium CNST to a solid plate M26 and cultured by a conventional method at 30° C. By 3-4 days later, the bacteria were collected, transferred to 50 mL of liquid medium M26, and cultured under rotation at 30° C. By 4-5 days later, the growth of the bacteria entered logarithmic growth phase. Subsequently, the bacteria was dispersed by a spinner bottle and transferred to another liquid medium M26 for amplification. Used as seeds for solid fermentation, the amplified bacteria were centrifuged, washed with sterile water, smeared on filter paper placed in a culture medium CNST, and cultured at 30° C. The bacteria entered logarithmic growth phase 3-4 days later. A layer of resin XAD16 (2%) was coated on the filter paper. After 7-9 days of culture at 30° C., the strain entered the secondary metabolism phase completely.

A formula of the medium CNST was: KNO₃ 0.5 g/L, Na₂HPO₄ 0.25 g/L, MgSO₄.7H₂O 1 g/L, FeCl₃ 0.001%, trace element solution 1 ml/L, agar 1.5%, pH 7.2. The formula was sterilized, a plate medium was prepared, and sterile filter paper was placed after cooling solidification.

A formula of the medium M26 was: potato starch 8 g/L, yeast extract powder 2 g/L, peptone 2 g/L, glucose 2 g/L, MgSO₄.7H₂O 1 g/L, CaCl₂ 1 g/L, EDTA-FeCl₃ 1 ml/L, trace element solution 1 ml/L, pH 7.2. Upon preparation of a solid medium, 12 g/L agar powder was needed.

A formula of the trace element solution was MnCl₂.4H₂O 0.1 g/L, CoCl₂ 0.02 g/L, CuSO₄ 0.01 g/L, Na₂MoO₄.2H₂O 0.01 g/L, ZnCl₂ 0.02 g/L, LiCl 0.005 g/L, SnCl₂.2H₂O 0.005 g/L, H₃BO₃ 0.01 g/L, KBr 0.02 g/L, and KI 0.02 g/L.

After 7-9 days of culture, the solid plate was collected, Sorangium cellulosum So0157-2 and the resin XAD were scraped by a sterile shovel, and the filter paper degraded by Sorangium cellulosum was collected by tweezers. The collected samples were placed in an oven (40° C.) for removal of excess water and immersed with methanol. The resultant immersion solution was filtered with filter paper and dried at 40° C. to yield an extract comprised of epothilone glycoside A-1, A-2, B-1, B-2, C-1, and C-2.

10 L of fermented Sorangium cellulosum So0157-2, CCTCC NO: M 208078 and the resin XAD were immersed with methanol. The resultant immersion solution was filtered with filter paper and dried at 40° C. to yield 1.25 g of extract comprising epothilone glycoside A-1, A-2, B-1, B-2, C-1, and C-2.

1.2 The Isolation and Purification of Epothilone Glycoside A-1

The fermentation extract of Sorangium cellulosum So0157-2, CCTCC NO: M 208078 was isolated by medium-pressure liquid chromatography (RP-18, 80 g), eluted with a methanol-water system, i.e., 50% 1500 mL to yield M1+M2 (940 mg), 65% 700 mL (M3, 100 mg), 75% 700 mL (M4, 250 mg), and eluted with 300 mL of methanol (M5, 82 mg). The eluates were measured by TLC and developed with petroleum ether-acetone (3:2). The eluates M3 and M4 had spots that could be colored by an alkaloid reagent, and the spot from the M3 had large polarity.

The eluate M3 was isolated with gel column chromatography and eluted with methanol. The resultant eluates were collected automatically with each tube of about 3 mL (2,600 seconds), measured by TLC, and developed with chloroform-methanol (10:1). The eluates 17-22 (44 mg) and 23-24 (11 mg) were combined, respectively. The eluates 17-22 was further isolated with gel column chromatography and eluted with methanol. The resultant eluates were collected automatically with each tube of about 3 mL (2,600 seconds), measured by TLC, and the eluates 5-8 (35 mg) were combined. The combined eluate was isolated with normal phase column chromatography, i.e., the chromatographic column was saturated with 0.8 g of silicone petroleum ether, and the sample was dissolved with chloroform, loaded, eluted with petroleum ether-ethyl acetate (10:1, 41 mL; 5:1, 48 mL) and chloroform-methanol (30:1) separately to yield a main component (25 mg). The main component was further isolated with normal phase column chromatography, i.e., the chromatographic column was saturated with 0.6 g of silicone petroleum ether, and the sample was dissolved with chloroform, loaded, eluted as a gradient with chloroform-methanol (40:1, 41 mL; 35:1, 36 mL; 30:1, 31 mL). 3-4 mL/tube of eluates were collected, measured by TLC, and the eluates 4-6 (13 mg) and 7-10 (5 mg) were combined, respectively. The combined eluate 7-10 was measured by TLC and developed with a variety of developers to yield a single spot, which showed that a pure compound was obtained. The compound was designated with the code EPO-E (NMR spectroscopy was measured with CDCl₃ as solvent).

### 2. The Structural Identification of the Compound EPO-E

ESI-MS showed the quasi-molecular ion peak of the compound EPO-E was I/1/z 626.4 [M+H]⁺ and 648.3 [M+Na]⁺, so the molecular weight of the compound was 625. The compound had a fragment peak m/z 492 after m/z 133 split off. High resolution fast atom bombardment mass spectrometry showed the formula of the compound was C₃₁H₄₇NO₁₀S (HRFAB-MS, measured value: m/z 625.7706, calculated value: m/z 625.2921).

C-NMR (comprising DEPT) of the compound EPO-E had 31 signals, comprising 6 methyl, 7 methylene, 12 methine, and 6 quaternary carbon. Based on the signals of ¹H NMR spectrum at δ 5.21 (s, H-1′, the anomeric proton), 3.91 (s, H-2′), 4.00 (m, H-3′), 4.31 (m, H-4′), 3.85 (H-5′), and 3.78 (dd, H-5′), the signals of ¹³C NMR spectrum at δ 108.7 (C-1′), 79.0 (C-2′), 78.2 (C-3′), 88.1 (C-4′), and 66.2 (C-5′), the signals of HMQC spectrum, and the signals of HMBC spectrum, a unit of α-D-ribofuranosyl was determined. Further studies on the signals of the HMQC spectrum and the HMBC spectrum showed that the compound was epothilone A (corresponding data are listed in Table 1). Based on the long-range correlation between the C-1′ proton and C-3, it was determined that the C-3 of epothilone A was substituted with a glycoside. Thus, the compound EPO-E was epothilone glycoside, a novel compound, named as epothilone glycoside A-1.

TABLE 1
NMR data of the compound EPO-E

| Number | ¹H^{b} | ¹³C | HMBC |
|---|---|---|---|
| 1 | | 170.2s | |
| 2 | 2.59 (m) | 38.5t | C-1, C-3, C-4 |
| 3 | 4.21 (dd, 3.2, 9.4) | 79.1d | |
| 4 | | 52.9s | |
| 5 | | 219.5s | |
| 6 | 3.15 (dq, 4.7, 6.8) | 43.8d | C-25, C-8, C-5 |
| 7 | 3.78 (t, 4.6) | 74.5d | C-25, C-28, C-9, C-8, C-6, C-5 |
| 8 | 1.70 (m, 2H) | 36.2t | |
| 9 | 1.45 (m, 2H) | 31.9t | |
| 10 | 1.62 (m, 2H) | 23.3t | C-12 |
| 11 | 1.86 (m), 1.46 (m) | 27.4t | |
| 12 | 2.95 (dt, 3.2, 9.1) | 57.4d | |
| 13 | 3.07 (dt, 3.5, 9.4) | 54.8d | C-12 |
| 14 | 2.14 (m), 1.94 (m) | 32.0t | C-14, C-27, C13, C-15 |
| 15 | 5.48 (dd, 1.7, 8.6) | 77.9d | C-27, C-14, C-13, C-17, C-1 |
| 17 | | 137.5s | |
| 18 | 6.61 (s) | 117.0d | C-17, C-15, C-27 |
| 19 | | 151.5s | |
| 20 | 7.00 (s) | 121.4d | C-21, C-19 |
| 21 | | 166.0s | |
| 22 | 2.71 (s) | 19.0q | C-21, C-19, |
| 23 | 1.15 (s) | 20.1q | C-24, C-4, C-5, C-3 |
| 24 | 1.36 (s) | 21.7q | C-23, C-4, C-5, C-3 |
| 25 | 1.18 (d, 6.8) | 14.1q | C-6, C-7 |
| 26 | 1.05 (d, 7.0) | 17.3q | C-9, C-8, C-7 |
| 27 | 2.08 (s) | 15.1q | C-15 |
| 1′ | 5.21 (s) | 108.7d | C-3, C-4′ |
| 2′ | 3.91 (br s) | 79.0d | C-3′, C-4′ |
| 3′ | 3.98 (br s) | 78.2d | C-4′ |
| 4′ | 4.31 (q, 2.2) | 88.1d | C-2′ |
| 5′ | 3.87 (dd, 2.7, 12.0) | 62.2t | C-2′, C-3′ |
| | 3.79 (dd, 2.2, 12.0) | | |

The data of ¹H, ¹³C NMR, and HMBC were measured at room temperature, with CDCl₃ as solvent, and at 600 MHz, 150 MHz, and 600 MHz, respectively.

In Table 1, without extra explanation, the proton signals (¹H^{b}) were normalized as ¹H.

### 3. Experiments of Antitumor Activity of Epothilone Glycoside A-1

Screening method: methyl-thiazol-tettazolium (MTT) reduction method and sulforhodamine B (SRB) protein staining method.

Cell strains: human liver cancer cell HepG2, human lung cancer cell A-549, and breast cancer cell MDA-MB-435.

Reaction time: 48-72 hrs

Results:

No effect: 10⁻⁵ mol/L<85%

Weak effect: 10⁻⁵ mol/L 85% or 10⁻⁶ mol/L>50%

Strong effect: 10⁻⁶ mol/L>85% or 10⁻⁷ mol/L>50%

The detailed results are listed in Table 2.

TABLE 2
Inhibition rate of growth of epothilone glycoside A-1 on human liver cancer
cell HepG2, human lung cancer cell A-549, and breast cancer cell MDA-MB-435

| | Concentration (M) | | | | | | |
|---|---|---|---|---|---|---|---|
| | 10⁻⁴ | 10⁻⁵ | 10⁻⁶ | 10⁻⁷ | 10⁻⁸ | IC₅₀ (μM) | 95% confidence limit |
| Cell strain | HepG2 | | | | | | |
| Inhibition rate of | 100 | 99.9 | 93.3 | 41.5 | 15.7 | 0.07 | 0.03-0.15 |
| growth | | | | | | | |
| Cell strain | A-549 | | | | | | |
| Inhibition rate of | 90.6 | 83.7 | 72.0 | 0 | 0 | 6.47 | 0.56-75.29 |
| growth | | | | | | | |
| Cell strain | MDA-MB-435 | | | | | | |
| | Concentration (μM) | | | | | | |
| | 1 | 2 | 4 | 8 | 10 | IC₅₀ (μM) | 95% confidence limit |
| Inhibition rate of | 18.9 | 55.0 | 76.1 | 84.6 | 98.2 | 2.07 | 0.18-33.6 |
| growth | | | | | | | |

Conclusion: as shown in Table 2, at the concentration of 10⁻⁶ M, epothilone glycoside A-1 had strong inhibition on human liver cancer cell HepG2 and weak inhibition on human lung cancer cell A-549, and human breast cancer cells MDA-MB-435. Therefore, the compound can selectively inhibit cancer cells, and the active site of the compound can function as a chemical inhibitor of cancer.

### Example 2

| Dosage form | Component | Mass |
|---|---|---|
| Tablet | Epothilone glycoside A-1 | 1 mg |
| | Lactose | 182 mg |
| | Cornstarch | 54 mg |
| | Magnesium stearate | 3 mg |

Preparation method: Epothilone glycoside A-1 was mixed with lactose and cornstarch. The resultant mixture was uniformly wet with water, screened, dried, screened again, magnesium stearate added, and made into tablet form. Each tablet was 240 mg with 1 mg of epothilone glycoside A-1.

### Example 3

| Dosage form | Component | Mass |
|---|---|---|
| Capsule | Epothilone glycoside A-1 | 1 mg |
| | Lactose | 197 mg |
| | Magnesium stearate | 2 mg |

Preparation method: Epothilone glycoside A-1 was mixed with lactose and magnesium stearate. The resultant mixture was screened, mixed uniformly, and packed into a hard gelatin capsule. Each capsule was 200 mg with 1 mg of epothilone glycoside A-1.

### Example 4

| Dosage form | Component | Mass |
|---|---|---|
| Ampoule | Epothilone glycoside A-1 | 1 mg |
| | NaCl | 9 mg |

Preparation method: Epothilone glycoside A-1 and NaCl were dissolved in water for injection. The resultant solution was filtered and packed in an ampoule under aseptic conditions.

### Example 5

| Dosage form | Component | Mass |
|---|---|---|
| Capsule | Epothilone glycoside B-1 | 1 mg |
| | Lactose | 197 mg |
| | Magnesium stearate | 2 mg |

Preparation method: Epothilone glycoside B-1 was mixed with lactose and magnesium stearate. The resultant mixture was screened, mixed uniformly, and packed into a hard gelatin capsule. Each capsule was 200 mg with 1 mg of epothilone glycoside B-1.

### Example 6

| Dosage form | Component | Mass |
|---|---|---|
| Ampoule | Epothilone glycoside B-1 | 1 mg |
| | NaCl | 9 mg |

Preparation method: Epothilone glycoside B-1 and NaCl were dissolved in water for injection. The resultant solution was filtered and packed in an ampoule under aseptic conditions.

### Example 7

| Dosage form | Component | Mass |
|---|---|---|
| Tablet | Epothilone glycoside C-1 | 1 mg |
| | Lactose | 182 mg |
| | Cornstarch | 54 mg |
| | Magnesium stearate | 3 mg |

Preparation method: Epothilone glycoside C-1 was mixed with lactose and cornstarch. The resultant mixture was uniformly wet with water, screened, dried, screened again, magnesium stearate added, and made into tablet form. Each tablet was 240 mg with 1 mg of epothilone glycoside C-1.

### Example 8

| Dosage form | Component | Mass |
|---|---|---|
| Capsule | Epothilone glycoside A-2 | 1 mg |
| | Lactose | 197 mg |
| | Magnesium stearate | 2 mg |

Preparation method: Epothilone glycoside A-2 was mixed with lactose and magnesium stearate. The resultant mixture was screened, mixed uniformly, and packed into a hard gelatin capsule. Each capsule was 200 mg with 1 mg of epothilone glycoside A-2.

### Example 9

| Dosage form | Component | Mass |
|---|---|---|
| Ampoule | Epothilone glycoside B-2 | 1 mg |
| | NaCl | 9 mg |

Preparation method: Epothilone glycoside B-2 and NaCl were dissolved in water for injection. The resultant solution was filtered and packed in an ampoule under aseptic conditions.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An epothilone glycoside having anticancer activity, said epothilone glycoside having a formula of 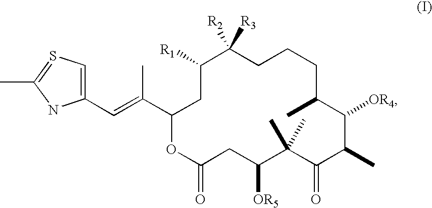
wherein

| | |
|---|---|
| R₁ + R₂ = O, R₃ = R₄ = H, R₅ = glycosyl | epothilone glycoside A-1 |
| R₁ + R₂ = O, R₃ = R₅ = H, R₄ = glycosyl | epothilone glycoside A-2 |
| R₁ + R₂ = O, R₃ = CH₃, R₄ = H, R₅ = glycosyl | epothilone glycoside B-1 |
| R₁ + R₂ = O, R₃ = CH₃, R₅ = H, R₄ = glycosyl | epothilone glycoside B-2, |

or 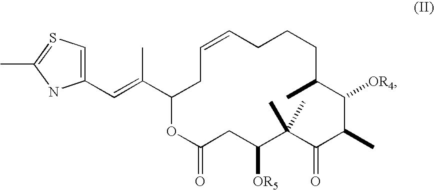
wherein

| | |
|---|---|
| R₄ = H, R₅ = glycosyl | epothilone glycoside C-1 |
| R₄ = glycosyl, R₅ = H | epothilone glycoside C-2. |

2. The epothilone glycoside of claim 1, wherein said epothilone glycoside is isolated from a solid or liquid fermentation product of Sorangium cellulosum So0157-2 CCTCC NO: M 208078.

3. A pharmaceutical composition comprising a therapeutically effective amount of epothilone glycoside of claim 1 and a pharmaceutically acceptable excipient for the treatment and prevention of cancer.

4. The pharmaceutical composition of claim 3, wherein said cancer is liver cancer.

5. The pharmaceutical composition of claim 3, wherein said cancer is lung cancer.

6. The pharmaceutical composition of claim 3, wherein said cancer is breast cancer.

7. The pharmaceutical composition of claim 3, wherein said epothilone glycoside accounts for between 0.1 and 99.5 wt. % of said pharmaceutical composition.

8. The pharmaceutical composition of claim 7, wherein said epothilone glycoside accounts for between 0.5 and 95 wt. % of said pharmaceutical composition.

9. The pharmaceutical composition of claim 3, wherein a daily dose of said epothilone glycoside is between 0.01 and 10 mg/Kg of body weight.

10. The pharmaceutical composition of claim 9, wherein a daily dose of said epothilone glycoside is between 0.1 and 5 mg/Kg of body weight.

11. The pharmaceutical composition of claim 3, wherein an administration mode of said pharmaceutical composition is oral administration, nasal inhalation, rectal administration, or parenteral administration.

12. A method for treatment and prevention of cancer comprising administering a patient in need thereof an epothilone glycoside of claim 1.

13. The method of claim 12, wherein said cancer is liver cancer.

14. The method of claim 12, wherein said cancer is lung cancer.

15. The method of claim 12, wherein said cancer is breast cancer.

16. The method of claim 12, wherein a daily dose of said epothilone glycoside is between 0.01 and 10 mg/Kg of body weight.

17. The method of claim 16, wherein a daily dose of said epothilone glycoside is between 0.1 and 5 mg/Kg of body weight.

18. The method of claim 12, wherein an administration mode of said epothilone glycoside is oral administration, nasal inhalation, rectal administration, or parenteral administration.
